(19) United States
(12) Patent Application Publication
(10) Pub. No.: US 2008/0095754 A1
(43) Pub. Date: Apr. 24, 2008

(54) OPHTHALMIC COMPOSITIONS COMPRISING DIGLYCINE

(76) Inventors: Susan E. Burke, Batavia, NY (US); Erning Xia, Penfield, NY (US); Kai Kwok, Rochester, NY (US); Stephen R. Davio, Fairport, NY (US)

Correspondence Address: Joseph Barrera;Bausch & Lomb Incorporated, One Bausch & Lomb Place, Rochester, NY, 14604, US

(21) Appl. No.: 11/854,608

(22) Filed: Sep. 13, 2007

Related U.S. Application Data
(60) Provisional application No. 60/852,487, filed on Oct. 18, 2006.

(51) Int. Cl. A61K 31/195; A61K 38/40; A61K 38/47; A61P 27/04; C11D 3/48

(52) U.S. Cl. 424/94.61; 510/114; 514/12; 514/563

(57) ABSTRACT

An ophthalmic composition that includes an epithelium cell stabilizer component. The epithelium cell stabilizer component may be one or more of diglycine, triglycine, tetraglycine, pentaglycine. The invention is also directed to a method of treating or preventing dry eye comprising administering an effective amount of the ophthalmic composition to the eye.

This application claims priority to U.S. provisional application Ser. No. 60/852,487.

### FIELD OF THE INVENTION

The invention relates to an ophthalmic composition comprising at least one epithelium cell stabilizer.

### BACKGROUND OF THE INVENTION

Dry eye, also known generically as keratoconjunctivitis sicca and dyslacrima, is a common ophthalmological disorder affecting millions of people. A patient with dry eye may experience burning, a feeling of dryness and persistent irritation. In severe cases, dry eye can seriously impair a person's vision and hence handicap the sufferer in activities such as driving. Certain diseases, such a Sjogren's disease, manifest dry eye symptoms. Also, as people age, the lacrimal glands in the eye may produce less moisture, resulting in eyes that become dry, inflamed, itchy and gritty.

Although it appears that dry eye may result from a variety of underlying, unrelated pathogenic causes, all presentations of the condition share a common effect, namely the breakdown of the pre-ocular tear film, which commonly results in dehydration of the exposed outer surface and hence the symptoms described above.

A number of approaches exist for the treatment of dry eye. One common approach has been to supplement the ocular tear film using artificial tears instilled throughout the day. Examples of the tear substitute approach include the use of buffered, isotonic saline solutions and aqueous solutions containing water-soluble polymers that render the solutions more viscous and thus less easily shed by the eye by the washing action of the tear fluid. See, for example, U.S. Pat. No. 5,209,927 to Gressel, et al.; U.S. Pat. No. 5,294,607 to Glonek, et al.; and U.S. Pat. No. 4,409,205 to Shively.

Although these approaches have met with some success in some cases, significant challenges in the treatment of dry eye nevertheless remain. Increasing the viscosity of the dry eye product may extend the product's duration in the eye, however, the overall benefit is somewhat limited.

### BRIEF SUMMARY OF THE INVENTION

The invention is directed to an ophthalmic composition that includes an epithelium cell stabilizer component. The epithelium cell stabilizer component may be one or more of diglycine, triglycine, tetraglycine, pentaglycine.

The invention is also directed to a method of treating or preventing dry eye comprising administering an effective amount of the ophthalmic composition to the eye.

### BRIEF DESCRIPTION OF THE SEVERAL VIEWS OF THE DRAWINGS

A detailed description of the invention is described with specific reference being made to the drawings.

FIG. 1 is a graph of DSC Thermograms of lysosyme in borate buffer.

FIG. 2 is a graph of DSC Thermograms of lysozyme in borate buffer with 1% diglycine.

FIG. 3 is a graph of DSC Thermograms of lysozyme in borate buffer with 3% diglycine.

FIG. 4 is a CD graph of the melting curve of lysozyme in borate buffer as a function of temperature.

FIG. 5 is a CD graph of the melting curve of lysozyme in borate buffer with 1% diglycine as a function of temperature.

FIG. 6 is a CD graph of the melting curve of lysozyme in borate buffer with 3% diglycine as a function of temperature.

FIG. 7 is a graph of DSC Thermograms of lysozyme in borate buffer with 1% glycerin.

FIG. 8 is a graph of DSC Thermograms of lysozyme in borate buffer with 1% trehalose.

FIG. 9 is a graph of DSC Thermograms of lysozyme in borate buffer with 5% trehalose.

FIG. 10 is a graph of DSC Thermograms of lysozyme in borate buffer with 1% diglycine and 3% glycerin.

FIG. 11 is a graph of DSC Thermograms of lysozyme in borate buffer with 1% diglycine and 3% trehalose.

FIG. 12 is a graph of DSC Thermograms of lysozyme in borate buffer with 1% diglycine and 0.2% hyaluronic acid.

FIG. 13 is a CD graph of the melting curve of lysozyme in borate buffer with 1% trehalose.

FIG. 14 is a CD graph of the melting curve of lysozyme in borate buffer with 5% trehalose.

FIG. 15 is a CD graph of the melting curve of lysozyme in borate buffer with 1% glycerin.

FIG. 16 is a CD graph of the melting curve of lysozyme in 1% diglycine and 3% glycerin.

FIG. 17 is a CD graph of the melting curve of lysozyme in 1% diglycine and 3% trehalose.

FIG. 18 is a CD graph of the melting curve of lysozyme in 1% diglycine and 0.2% hyaluronic acid.

### DETAILED DESCRIPTION OF THE INVENTION

The normal conjunctiva and cornea are protected by a triple-layered tear film comprising an outer oily layer from the meibomian glands, an aqueous layer from lacrimal glands and an inner layer of mucus, derived mainly from conjunctival goblet cells. Tears produced by the lacrimal glands have a high concentration of IgA, lysozyme and lactoferrin are known to have antimicrobial activity. This antimicrobial activity is likely the result of the coating of bacteria by tear IgA, growth inhibition by lactoferrin iron chelation and the lytic action of lysozyme. Lysozyme splits the bond between acetylmuramic acid and acetylglucosamine in the peptidoglycan of the bacterial cell wall with a direct lytic action.

A stable tear film can be critical to prevent pathogenic microorganism invasion. Microorganism invasion can be facilitated by an epithelial defect, unstable tear film, or contaminated contact lenses. A stable preocular tear film depends on many factors, including the correct quantity and quality of various components of the tears and the integrity of the corneal epithelium. Environmental pollution, smoking and frequent use of eye drops can cause denaturization of tear proteins such as lysozyme and lactoferrin. The denatured tear proteins can cause destabilization of tear film, staining, loss of tight junction, and dry eye.

The ophthalmic composition includes an at least one epithelium cell stabilizer selected from the group consisting of diglycine, glycine, triglycine, tetraglycine and pentaglycine. The epithelium cell stabilizer is generally present in the composition at a concentration of from 0.001% w/w to a 10% w/w, for instance 0.1% w/w to 5% w/w or 0.1% w/w to 2% w/w.

As used in this application, “ophthalmic composition” is defined as a composition intended for application in the eye or intended for treating a device to be placed in contact with the eye such as a contact lens. Ophthalmic compositions can include compositions for direct placement in the eye and include eye drop and eye wash solutions such as for treating dry eye. Ophthalmic compositions also include those compositions formulated as multi-purpose solutions for cleaning and disinfecting contact lenses or to package contact lenses.

The ophthalmic composition can also include one or more polyols. Examples of polyols include, but are not limited to, glycerine, trehalose, arabitol, erythirtol, glycerol, lactitol, maltitol, mannitol, sorbitol, xylitol.

The ophthalmic compositions can also include a tear protein. For instance, lysozyme, lactoferrin or any combination thereof can be present in the composition in an amount of from 0.001% w/w to 3% w/w, for example from 0.1 w/w % to 1% w/w.

The ophthalmic composition can also include at least one preservative. Examples of suitable preservatives include, but are not limited to, benzalkonium chloride (BAK), benzalkonium chloride (BAK)/ethylenediaminetetraacetic acid (EDTA), sorbic/ethylenediaminetetraacetic acid (EDTA), biguanides, sodium perborate and hydrogen peroxide.

The ophthalmic composition can also include one or more therapeutic agents. Therapeutic agents include anti-inflammatory agents, antibiotics, antimicrobial components, immunosuppressive agents, antifungal agents, antiprotozoal agents and any combination thereof.

Non limiting examples of anti-inflammatory agents include glucocorticosteroids (e.g., for short term treatment) and non-steroidal anti-inflammatory drugs or NSAIDs.

Non-limiting examples of glucocorticosteroids are: 21-acetoxypregnenolone, alclometasone, algestone, emcinonide, beclomethason, betamethasone, budesonide, chloroprednisone, clobetasol, clobestasone, clocortolone, cloprednol, corticosterone, cortisone, cortivazol, deflazacort, desonide, desoximetasone, dexamethasone, diflorasone, diflurcortolone, difluprednate, enoxolone, fluazacort, clucloronide, flumethasone, flunisolide, fluocinolone acetonide, fluocinonide, fluocorin butyl, fluorcortolong, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, their physiologically acceptable salts, derivatives thereof, combinations thereof, and mixtures thereof. In at least one embodiment, the therapeutic agent is selected from the group consisting of difluprednate, loteprednol etabonate, prednisolone and any combination thereof.

Non-limiting examples of antibiotics include doxorubicin; aminoglycosides (e.g., amikacin, apramycin, arbekacin, bambermycins, butirosin, dibekacin, dihydrostrptomycin, fortimicin(s), gentmicin, isepemacin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin SV, rifapentine, rifaximin), β-lactams (e.g., carbacephems (e.g., loracarbef)), carbapenems (e.g., biapenem, imipenem, meropenem, panipenem), cephalosporins (e.g., cefaclor, cefadroxil, cefamandole, cefatrizine, cefazedone, defazolin, cefcapene pivoxil, cefclidin, cefdinir, cefditoren, cefepime, cefetamet, cefixime, cefinenoxime, cefodizime, cefonicid, cefoperzone, ceformide, cefotzxime, cefotiam, cefozopran, cefpimizole, cefpiramide, cefpirome, cefpodoxime proxetil, cefprozil, cefroxzdine, cefsulodin, ceftazidime, defteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephalexin, cephaloglycin, cephaloridine, cephalosporin, cephalothin, cephapirin sodium, ceplradine, pivcefalexin), cephamycins (e.g., cefbuperazone, cefinetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benxylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, ticarcillin), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin S, gramicidin(s), mikamycin, polymyxin, pristinamycin, ristocetin, teicoplanin, thiostrpton, tuberactinomycin, tyrocidine, tyrothricin, bancomycin, ciomycin, virginiamycin, zinc bacitracin), tretracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin).

Other examples of antibiotics are the synthetic antibacterials, such as 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benxylsulfamide, chloramine-B, chloramine-T, dichloramine T, n²-formylsulfisomidine, n⁴-β-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, n⁴-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole), sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene citrate, methenamine hippurate, methenamine mandelate, methenamine sulfosalicylate, nitroxoline, taurolidine, xibomol).

Non-limiting examples of immunosuppressive agents include dexamethasone, cyclosporin A, azathioprine brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thiaguanine), pyrimidine analogs (e.g., ancitabine, azacitidine, 6-azauridine, cannofur, cytarabine, doxifluridine, emitefur, enocitabine, floxuridine, fluorouracil, gemcitabine, tegafur), fluocinolone, triaminolone, anecortave acetate, fluorometholone, medrysone, and prednisolone.

Non-limiting examples of antifungal agents include polyenes (e.g., amphotericin B, candicidin, dermostatin, filipin, fungichromin, hachimycin, hamycin, lucensomycin, mepartricin, natamycin, nystatin, pecilocin, perimycin), azaserine, griseofulvin, oligomycins, neomycin undecylenate, pyirolnitrin, siccanin, tubercidin, viridian, allylamines (e.g., butenafine, naftifine, terbinafine), imidazoles (e.g., bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, golnaftate), triazoles (e.g., fluconazole, itraconazole, saperconazole, terconazole), acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, and zinc propionate.

The ophthalmic compositions can be formulated as a contact lens solution to disinfect, clean or package contact lenses. In such applications, the compositions will include one or more cationic antimicrobial components. The term “cationic” when referring to an antimicrobial component refers to the predominant form of the antimicrobial component at neutral pH having a positive charge and a counteranion. Suitable cationic antimicrobial components are those generally used in contact lens solutions and include, but are not limited to, quaternary ammonium salts such as α-[4-tris(2-hydroxyethyl)ammonium chloride-2-butenyl]poly[1-dimethylammonium chloride-2-butenyl]-ω-tris(2-hydroxyethyl)ammonium chloride (available as polyquaternium-1 from Stepan Corporation), benzalkonium halides, and biguanides such as salts of alexidine, alexidine-free base, salts of chlorhexidine, hexamethylene biguanides and salts thereof and their polymers such as poly(hexamethylene biguanide) (PHMB) or PHMB-CG*. An exemplary list of cationic disinfecting antimicrobial components include α-[4-tris(2-hydroxyethyl)ammonium chloride-2-butenyl]poly[1-dimethylammonium chloride-2-butenyl]-ω-tris(2-hydroxyethyl)ammonium chloride, poly(hexamethylene biguanide) (PHMB), PHMB-CG* and any combination thereof.

A new synthetic route to polymeric biguanide compositions is described in copending U.S. provisional application Ser. No. 60/853,579 filed Oct. 23, 2006, and 60/895,770 filed Mar. 20, 2007, hereby incorporated by reference herein in its entirety. The new synthetic route provides a polymeric biguanide composition comprising less than 18 mol % of terminal amine groups as measured by ¹³C NMR. The polymeric biguanide composition also is characterized by a relative increase in the molar concentration of terminal guanidine groups or terminal cyanoguanidino groups. For example, in at least one embodiment, the biguanide composition comprises less than 18 mol % of terminal amine groups, and 55 mol % or greater of terminal guanidine groups. In at least one embodiment, the biguanide composition comprises less than 18 mol % of terminal amine groups, and 40 mol % or greater of terminal cyanoguanidino groups.

The antimicrobial component is generally present in an amount from 0.01 ppm to 50 ppm, from 0.1 ppm to 15 ppm or from 0.1 ppm to 10 ppm. It is preferred, however, that the amount of antimicrobial component that is used is effective in disinfecting contact lenses contacted with the compositions, while at the same time promote lens patient comfort and acceptability.

In some embodiments the primary antimicrobial component present in the lens care solutions may be hexamethylene biguanide, which is present from 0.2 ppm to 2 ppm. In other embodiments, the primary antimicrobial component present in the lens care solution may be α-[4-tris(2-hydroxyethyl)ammonium chloride-2-butenyl]poly[1-dimethylammonium chloride-2-butenyl]-ω-tris(2-hydroxyethyl)ammonium chloride, which is present from 1 ppm to 15 ppm.

In addition, any one mixture of two antimicrobial components can be present in the lens care solutions. For example, a particular lens care solution can include from 0.3 ppm to 0.8 ppm of a hexamethylene biguanide, and 3 ppm to 10 ppm α-[4-tris(2-hydroxyethyl)ammonium chloride-2-butenyl]poly[1-dimethylammonium chloride-2-butenyl]-ω-tris(2-hydroxyethyl)ammonium chloride.

The ophthalmic composition can also include a fatty acid monoester. The fatty acid monoester comprises an aliphatic fatty acid portion having ten carbon atoms, and an aliphatic hydroxyl portion. In some instances, and depending upon the particular type of contact lens, the presence of the fatty acid monoester can enhance the efficacy against Candida albicans or Fusarium solani.

The ophthalmic compositions can also include a phosphonic acid, or its physiologically compatible salt, represented by the following formula:

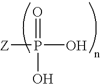

wherein Z is a connecting radical equal, n is an integer from 1 to 4, or 1, 2 or 3, and preferably containing 1 to 12 carbon atoms, more preferably 3 to 10 carbon atoms. The Z radical comprises substituted or unsubstituted saturated hydrocarbon radicals or amine-containing radicals, which amine-containing radicals are saturated hydrocarbon radicals in which the carbon atoms are interrupted with at least one nitrogen atom such as 1, 2 or 3 nitrogen atoms that forms a secondary or tertiary amine.

Accordingly, suitable Z radicals include substituted or unsubstituted alkylidene, substituted or unsubstituted alkylene, amino tri(alkylene) having at least n+1 carbon atoms, amino di(alkylene) having at least n+1 carbon atoms, alkylenediaminetetra(alkylene) or a dialkylenetriamine penta(alkylene) radical. In each case, the alkylene group in parenthesis is connected to a phosphonic acid group. Preferably, all alkylene groups independently have 1 to 4 carbon atoms.

Exemplary compounds in which the Z group is an amino tri(alkylene) radical includes amino tri(ethylidene phosphonic acid), amino tri(isopropylidene phosphonic acid), amino di(methylene phosphonic acid) mono(isopropylidene phosphonic acid), and amino mono(methylene phosphonic acid) di(ethylidene phosphonic acid). Exemplary compounds in which the Z group is a substituted or unsubstituted alkylidene radical includes methylene diphosphonic acid, ethylidine diphosphonic acid, 1-hydroxy propylidene diphosphonic acid. Exemplary compounds in which the Z group is an alkylenediaminetetra(alkylene) or a dialkylenetriamine penta(alkylene) radical include hexamethylenediaminetetra(methylene phosphonic acid) and diethylenetriaminepenta(methylenephosphonic acid).

In some embodiments, the phosphonic acid, or its physiologically compatible salt, may be represented by the following formula:

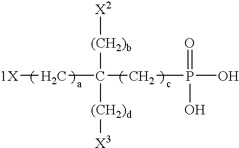

wherein each of a, b, c, and d are independently selected from integers from 0 to 4, preferably 0 or 1; X¹ is a phosphonic acid group (i.e., P(OH)₂O), hydroxy, amine or hydrogen; and X² and X³ are independently selected from the group consisting of halogen, hydroxy, amine, carboxy, alkylcarbonyl, alkoxycarbonyl, or substituted or unsubstituted phenyl, and methyl. Exemplary substituents on the phenyl are halogen, hydroxy, amine, carboxy and/or alkyl groups. A particularly preferred species is that wherein a, b, c, and d in are zero, specifically the tetrasodium salt of 1-hydroxyethylidene-1,1-diphosphonic acid, also referred to as tetrasodium etidronate, commercially available from Monsanto Company as DeQuest® 2016 diphosphonic acid sodium salt or phosphonate.

The ophthalmic composition can also include dexpanthenol, which is an alcohol of pantothenic acid, also called Provitamin B5, D-pantothenyl alcohol or D-panthenol. In some formulations of the lens care compositions, dexpanthenol can exhibit good cleansing action and can stabilize the lachrymal film at the eye surface when placing a contact lens on the eye. Dexpanthenol is preferably present in the contact lens care compositions in an amount from 0.2% to 10% (w/v), from 0.5% to 5% (w/v), or from 1% to 3% (w/v).

The ophthalmic composition can also include sorbitol, which is a hexavalent sugar alcohol. Typically, dexpanthenol is used in combination with sorbitol. In specific formulations, the combination dexpanthenol and sorbitol can provide enhanced cleansing action and can also stabilize the lachrymal film following placement of the contact lens on the eye. These formulations can substantially improve patient comfort when wearing contact lenses. Sorbitol may be present in the lens care compositions in an amount from 0.4% to 6% (w/v), from 0.8% to 4% (w/v) or from 1% to 3% (w/v).

The ophthalmic composition can also include at least one oil or oily substance. Any suitable oil or oily substance or combinations of oils or oily substances can be used provided such oils do not cause any substantial or significant detrimental effect to the patient or to a contact lens. The oil component can be a natural or synthetic oil. Natural oils can be obtained from plants or plant parts such as seeds, or they may be obtained from an animal source such as Sperm Whale oil, Cod liver oil and the like. The oil may be a mono, di or triglyceride of fatty acids or mixtures of glycerides. The oil may also be comprised of straight chain monoethylene acids and alcohols in the form of esters such as Jojoba and Sperm Whale oil.

The ophthalmic composition can also include at least one or more neutral or basic amino acids. Non-limiting examples of neutral amino acids include the alkyl-group-containing amino acids such as alanine, isoleucine, valine, leucine and proline; hydroxyl-group-containing amino acids such as serine, threonine and 4-hydroxyproline; thio-group-containing amino acids such as cysteine, methionine and asparagine. Non-limiting examples of the basic amino acid include lysine, histidine and arginine. The one or more neutral or basic amino acids are present in the ophthalmic compositions at a total concentration of from 0.1% to 5% (w/v).

In U.S. Pat. No. 5,741,817 it is described that certain amino acids enhance the effectiveness of anti-microbial components in ophthalmic compositions. An even greater enhancement is described in the commonly owned application entitled “Ophthalmic Compositions Comprising Dipeptides,” Attorney Docket Number B104.2B-13620-US01, incorporated herein by reference in its entirety, for ophthalmic compositions containing an anti-microbial and a dipeptide that has one glycine unit and a non-glycine unit. Accordingly, the ophthalmic compositions may be formulated to also utilize such anti-microbial enhancement chemistry.

The ophthalmic composition can also include an oligopeptide in addition to diglycine, triglycine, tetraglycine and pentaglycine, for instance dipeptides composed of a glycine unit and a non-glycine unit, can also function as an epithelium cell stabilizer in the compositions.

The ophthalmic composition can also include glycolic acid, asparatic acid, or an a-hydroxy acid or any mixture thereof, at a total concentration of from 0.001% to 4% (w/v) or from 0.01% to 2.0% (w/v). In addition, the combined use of one or more amino acids and glycolic acid, asparatic acid or α-hydroxy acid can minimize the dimensional change of the contact lens due to swelling and shrinkage following placement of the lens on the eye. The stated combination provides a higher degree of compatibility with the contact lens.

The ophthalmic composition can also include 2-amino-2-methyl-1,3-propanediol or a salt thereof (AMPD). Preferably, the AMPD is added to the solutions in an amount to satisfy a predetermined molar ratio of glycolic acid, asparatic acid, α-hydroxy acid or any mixture thereof and AMPD. The molar ratio of glycolic acid, asparatic acid, α-hydroxy acid or any mixture thereof to AMPD is 1:20 to 1.3:1, from 1:15 to 1.2:1 or from 1:14 to 1:1. The glycolic acid, asparatic acid, α-hydroxy acid or any mixture thereof is present in the ophthalmic compositions at a concentration of 0.01% to 5% (w/v) or at a concentration of 0.05% to 1% (w/v).

The ophthalmic composition can also include a buffer system. As used in this application, the terms “buffer” or “buffer system” is meant a compound that, usually in combination with at least one other compound, provides a buffering system in solution that exhibits buffering capacity, that is, the capacity to neutralize, within limits, either acids or bases (alkali) with relatively little or no change in the original pH. Generally, the buffering components are present from 0.05% to 2.5% (w/v) or from 0.1% to 1.5% (w/v).

The term “buffering capacity” is defined to mean the millimoles (mM) of strong acid or base (or respectively, hydrogen or hydroxide ions) required to change the pH by one unit when added to one liter (a standard unit) of the buffer solution. The buffer capacity will depend on the type and concentration of the buffer components. The buffer capacity is typically provided in solutions having a starting pH in the range of 6 to 9.

Borate buffers include, for example, boric acid and its salts, for example, sodium borate or potassium borate. Borate buffers also include compounds such as potassium tetraborate or potassium metaborate that produce borate acid or its salt in solutions. Borate buffers are known for enhancing the efficacy of certain polymeric biguanides. See, U.S. Pat. No. 4,758,595 to Ogunbiyi et al.

A phosphate buffer system preferably includes one or more monobasic phosphates, dibasic phosphates and the like. Particularly useful phosphate buffers are those selected from phosphate salts of alkali and/or alkaline earth metals. Examples of suitable phosphate buffers include one or more of sodium dibasic phosphate (Na₂HPO₄), sodium monobasic phosphate (NaH₂PO₄) and potassium monobasic phosphate (KH₂PO₄). The phosphate buffer components frequently are used in amounts from 0.01% or to 0.5% (w/v), calculated as phosphate ion.

Other known buffer compounds can optionally be added to the lens care compositions, for example, citrates, sodium bicarbonate, TRIS, and the like. Other ingredients in the solution, while having other functions, may also affect the buffer capacity. For example, EDTA, often used as a complexing agent, can have a noticeable effect on the buffer capacity of a solution.

A preferred buffer system is based upon boric acid/borate, a mono and/or dibasic phosphate salt/phosphoric acid or a combined boric/phosphate buffer system. For example a combined boric/phosphate buffer system can be formulated from a mixture of sodium borate and phosphoric acid, or the combination of sodium borate and the monobasic phosphate.

In a combined boric/phosphate buffer system, the solution comprises about 0.05 to 2.5% (w/v) of a phosphoric acid or its salt and 0.1 to 5.0% (w/v) of boric acid or its salt. The phosphate buffer is used (in total) at a concentration of 0.004 to 0.2 M (Molar), preferably 0.04 to 0.1 M. The borate buffer (in total) is used at a concentration of 0.02 to 0.8 M, preferably 0.07 to 0.2 M.

The ophthalmic composition can also include a water-soluble borate-polyol complex which can be formed by mixing a source of borate with a polyol of choice in an aqueous solution. These complexes can be used in conjunction with the antimicrobial component above, and can help to meet preservative efficacy and disinfection standards. In such compositions, the molar ratio of borate to polyol is generally from 1:0.1 to 1:10, or from 1:0.25 to 1:2.5. If present in the lens care solutions, the borate-polyol complex is usually present from 0.5% to 5% (w/v), from 1.0% to 2.5% (w/v). The borate-polyol complexes are described in greater detail in U.S. Pat. No. 6,143,799.

In some embodiments, the ophthalmic composition includes effective amounts of one or more of the following formulation components; a surfactant component, a viscosity enhancing, inducing, or thickening component, a chelating or sequestering component, or a tonicity component. The additional component or components can be selected from materials which are known to be useful in contact lens care solutions and are included in amounts effective to provide the desired effect or benefit.

Examples of suitable surfactants include amphoteric, cationic, anionic, or nonionic surfactants. Surfactants are typically present (individually or in combination) in amounts up to 8%, or up to 3% (w/v). One preferred surfactant class is the amphoteric or nonionic surfactants. The surfactant should be soluble in the lens care solution and non-irritating to eye tissues. Many nonionic surfactants comprise one or more chains of polymeric components having oxyalkylene (—OR—) repeats units wherein R has 2 to 6 carbon atoms. Preferred non-ionic surfactants comprise block polymers of two or more different kinds of oxyalkylene repeat units, which ratio of different repeat units determines the HLB of the surfactant. Satisfactory non-ionic surfactants include polyethylene glycol esters of fatty acids, e.g. polysorbate. Examples of this class include polysorbate 20 (available under the trademark Tween® 20), polyoxyethylene (23) lauryl ether (Brij® 35), polyoxyethyene (40) stearate (Myrj® 52), polyoxyethylene (25) propylene glycol stearate (Atlas® G 2612). Still other preferred surfactants include tyloxapol, polysulfates, polyethylene glycol, alkyl esters and any mixture thereof.

A particular non-ionic surfactant consisting of a poly(oxypropylene)-poly(oxyethylene) adduct of ethylene diamine having a molecular weight from about 7,500 to about 27,000 wherein at least 40 weight percent of said adduct is poly(oxyethylene) has been found to be particularly advantageous for use in cleaning and conditioning both soft and hard contact lenses when used in amounts from about 0.01 to about 15 weight percent. The CTFA Cosmetic Ingredient Dictionary's adopted name for this group of surfactants is poloxamine. Such surfactants are available from BASF Wyandotte Corp., Wyandotte, Mich., under Tetronic®.

An analogous of series of surfactants, for use in the lens care compositions, is the poloxamer series which is a poly(oxyethylene)poly(oxypropylene) block polymers available under Pluronic® (commercially available form BASF). In accordance with one embodiment of a lens care composition the poly(oxyethylene)-poly(oxypropylene) block copolymers will have molecular weights from 2500 to 13,000 daltons or from 6000 to about 12,000 daltons. Specific examples of surfactants which are satisfactory include: poloxamer 108, poloxamer 188, poloxamer 237, poloxamer 238, poloxamer 288 and poloxamer 407. Particularly good results are obtained with poloxamer 237.

Various other ionic as well as amphoteric and anionic surfactants suitable for in the invention can be readily ascertained, in view of the foregoing description, from McCutcheon's Detergents and Emulsifiers, North American Edition, McCutcheon Division, MC Publishing Co., Glen Rock, N.J. 07452 and the CTFA International Cosmetic Ingredient Handbook, Published by The Cosmetic, Toiletry, and Fragrance Association, Washington, D.C.

Amphoteric surfactants suitable for use in a composition according to the present invention include materials of the type are offered commercially under the trade name “Miranol.” Another useful class of amphoteric surfactants is exemplified by cocoamidopropyl betaine, commercially available from various sources.

The foregoing surfactants will generally be present in a total amount from 0.01% to 5% (w/v), from 0.1% to 3% (w/v), or from 0.1% to 1.5% (w/v). Often the amount of surfactant is from 0.005% or 0.01%, to 0.1% or 0.5% or 0.8% (w/v).

The ophthalmic composition can also include a viscosity enhancing/inducing component. The viscosity inducing components should be compatible with the other components and are preferably nonionic. Such viscosity inducing components are effective to enhance and/or prolong the cleaning and wetting activity of the surfactant component and/or condition the lens surface rendering it more hydrophilic (less lipophilic) and/or to act as a demulcent on the eye. Increasing the solution viscosity provides a film on the lens which may facilitate comfortable wearing of the contact lens. The viscosity inducing component can also function to cushion the impact on the eye surface during placement of the lens and serves also to alleviate eye irritation.

Suitable viscosity inducing components include, but are not limited to, water soluble natural gums, cellulose-derived polymers and the like. Useful natural gums include guar gum, gum tragacanth and the like. Useful cellulose-derived viscosity inducing components include cellulose-derived polymers, such as hydroxypropyl cellulose, hydroxypropylmethyl cellulose, carboxymethyl cellulose, methyl cellulose, hydroxyethyl cellulose and the like. A very useful viscosity inducing component is hydroxypropylmethyl cellulose (HPMC). Another useful viscosity inducing component is a polymer comprising monomeric units of 2-methacryloyloxy ethyl phosphorylcholine (MPC), which is available under the tradename Lipidure® from NOF Corporation.

The ophthalmic compositions may include hyaluronic acid or physiologically compatible salts (hereafter, collectively as hyaluronic acid). Examples of salts include metal salts such as sodium hyaluronate, potassium hyaluronate, magnesium hyaluronate, and calcium hyaluronate. In its natural form, hyaluronic acid has a molecular weight in the range of 5×10⁴ up to 1×10⁷ daltons. Its molecular weight may be reduced via a number of cutting processes such as exposure to acid, heat (e.g. autoclave, microwave, dry heat), or ultrasonic waves.

The viscosity inducing component is used in an amount effective to increase the viscosity of the solution, preferably to a viscosity in the range of 1.5 to 30, or even as high as 750, cps at 25° C., as determined by USP test method No. 911 (USP 23, 1995).

The ophthalmic composition will typically include an effective amount of a tonicity adjusting component. Among the suitable tonicity adjusting components that can be used are those conventionally used in contact lens care products such as various inorganic salts. Polyols and polysaccharides can also be used to adjust tonicity. The amount of tonicity adjusting component is effective to provide an osmolality from 200 mOsmol/kg to 400 mOsmol/kg or from 260 mOsmol/kg to 350 mOsmol/kg.

The ophthalmic compositions can be formulated as eye drop solution for treating dry eye. In such case it is preferable that an osmolality thereof is adjusted at 260 mOsmol/kg to 350 mOsmol/kg and the pH is adjusted to the range of 5.0-8.0. The ophthalmic compositions can also be formulated as eye wash solutions.

The ophthalmic compositions can be formulated as a disinfecting/cleaning solution for contact lenses. In general a contact lens disinfecting/cleaning method would include contacting or soaking the lenses with the solution for a period of time, typically for a minimum of one to four hours. Although such contacting may be accomplished by simply soaking a lens in the ophthalmic composition, greater preserving, disinfecting and/or cleaning may possibly be achieved if a few drops of the solution are initially placed on each side of the lens, and the lens is rubbed for a period of time, for example, approximately 20 seconds. The lens can then be subsequently immersed within several milliliters of the solution. Preferably, the lens is permitted to soak in the solution for at least four hours. Furthermore, the lens is preferably rinsed with fresh composition after any rubbing step and again after being immersed within the solution. The lenses are removed from the solution, rinsed with the same or a different solution, for example, a preserved isotonic saline solution, and repositioned on the eye.

The ophthalmic compositions can also be formulated for use as a preservative solution or a packaging solution for contact lenses. One of ordinary skill in the art would know how to adjust the formulation for each of these respective applications. The lens care compositions in combination with its container or bottle and packaging, including instructions for use in accordance with a specified regimen, provides an improved kit, package, or system for the care of contact lenses.

The ophthalmic composition can be formulated for use with many different types of contact lenses including: (1) hard lenses formed from materials prepared by polymerization of acrylic esters, such as poly(methyl methacrylate) (PMMA), (2) rigid gas permeable (RGP) lenses formed from silicone acrylates and fluorosilicone methacrylates, (3) soft, hydrogel lenses, and (4) non-hydrogel elastomer lenses.

As an example, soft hydrogel contact lenses are made of a hydrogel polymeric material, a hydrogel being defined as a crosslinked polymeric system containing water in an equilibrium state. In general, hydrogels exhibit excellent biocompatibility properties, i.e., the property of being biologically or biochemically compatible by not producing a toxic, injurious or immunological response in a living tissue. Representative conventional hydrogel contact lens materials are made by polymerizing a monomer mixture comprising at least one hydrophilic monomer, such as (meth)acrylic acid, 2-hydroxyethyl methacrylate (HEMA), glyceryl methacrylate, N,N-dimethacrylamide, and N-vinylpyrrolidone (NVP). In the case of silicone hydrogels, the monomer mixture from which the copolymer is prepared further includes a silicone-containing monomer, in addition to the hydrophilic monomer. Generally, the monomer mixture will also include a crosslink monomer such as ethylene glycol dimethacrylate, tetraethylene glycol dimethacrylate, and methacryloxyethyl vinylcarbonate. Alternatively, either the silicone-containing monomer or the hydrophilic monomer may function as a crosslink agent.

Although the invention can be embodied as many different compositions as described above, the compositions described is an exemplification of the principles of the invention and is not intended to limit the invention to the particular embodiments illustrated.

The invention is illustrated by the following non-limiting examples.

### EXAMPLES

Formulations 1 and 2 are two ophthalmic compositions containing diglycine which were used to examine the ability of diglycine to act as an epithelium cell stabilizer in Experiment No. 1 and Experiment No. 2.

Formulation 1. Ophthalmic Composition Containing Diglycine

| Ingredient | % w/w |
|---|---|
| Diglycine | 1.00 |
| Sodium Borate | 0.09 |
| Boric Acid | 0.85 |
| Sodium Chloride | 0.22 |
| Lysozyme | 0.50 |

Formulation 2. Ophthalmic Composition Containing Diglycine

| Ingredient | % w/w |
|---|---|
| Diglycine | 3.00 |
| Sodium Borate | 0.09 |
| Boric Acid | 0.85 |
| Sodium Chloride | 0.22 |
| Lysozyme | 0.50 |

### Experiment No. 1. Evaluating the Effect of Diglycine on the Conformational Stability of Lysozyme

To evaluate the effect of diglycine on the conformational stability of lysozyme, Formulations 1 and 2 as well as a formulation without diglycine were analyzed by differential scanning calorimetry (DSC) and circular dichroism (CD). Before DSC and CD analysis, a lysozyme solution was prepared in borate buffer, pH 7.3 (sodium borate, 0.09%; boric acid, 0.85%; sodium chloride, 0.22%) at a concentration of 5 mg/mL, and was dialyzed against the formulated solutions containing 0, 1 (Formulation 1), and 3% (Formulation 2) diglycine, at 2-8° C. for 16 hours. Thermal scans were preformed twice to investigate the reversibility of thermal unfolding for both DSC and CD. This study examines the values of melting temperature (Tm), unfolding enthalpies (ΔH), and the ability of the lysozyme to refold upon unfolding for each of the three formulations because these parameters, in general, predict the thermostability of a protein and approximates the susceptibility of a protein to aggegrate and other irreversible changes at lower temperatures.

For the DSC analysis, thermograms were obtained on a VP-DSC (Microcal, USA) at a scan rate of 90° C. per hour from 10° C. to 95° C. All samples were diluted with the formulated solutions to a concentration of 0.5 mg/mL, and degassed for 4 minutes using a vacuum. The thermograms were corrected for the baseline signal and normalized for protein concentration. Using a non-2-state unfolding model, the Tm and the ΔH were determined by Origin software.

CD spectra were conducted on a Jasco-810 Spectropolarimeter. The far-UV CD studies were preformed in a 1-mm pathlength cell at a protein concentration of 0.2 mg/mL, using a scan rate of 50 nm/min from 190 nm to 260 nm and a scan rate of 1° C./min from 20° C. to 95° C. The melting temperatures and the unfolding enthalpies were determined from the denaturation curved obtained from the dependency of the mean residue ellipticity at 230 nm on temperature.

The results of the DSC and CD analysis are provided in Table 1. As can be seen in Table 1, the VP-DSC analysis showed that the Tm increased with increasing concentrations of diglycine. This indicates the native form of lysozyme exhibited greater conformation stability at a higher concentration of diglycine. The CD analysis showed that the native form of lysozyme showed significant stabilization in the presence of diglycine. In both analytical approaches, the addition of 1% w/w and 3% w/w diglycine led to an increase in the melting temperature of lysozyme by approximately 2° C. and 4° C., respectively.

TABLE 1
Effect of Diglycine on Tm and ΔH of Lysozyme by VP-DSC and CD

| Lysozyme | VP-DSC | | CD | |
|---|---|---|---|---|
| Formulated | ΔH (1 × 10⁵ | | ΔH (1 × 10⁵ | |
| Solutions | Cal/Mol) | Tm (° C.) | Cal/Mol) | Tm (° C.) |
| Borate Buffer | | | | |
| 1^{st} run | 1.106 | 72.43 | 1.125 | 72.95 |
| 2^{nd} run | N.D. | N.D. | N.D. | N.D. |
| Borate Buffer with | | | | |
| 1% Diglycine | | | | |
| 1^{st} run | 1.112 | 74.93 | 1.057 | 75.15 |
| 2^{nd} run | 0.8694 | 74.84 | 0.7601 | 75.13 |
| Borate Buffer with | | | | |
| 3% Diglycine | | | | |
| 1^{st} run | 1.091 | 76.67 | 1.164 | 77.05 |
| 2^{nd} run | 0.8811 | 76.52 | 0.8608 | 77.63 |

N.D = Not Detected

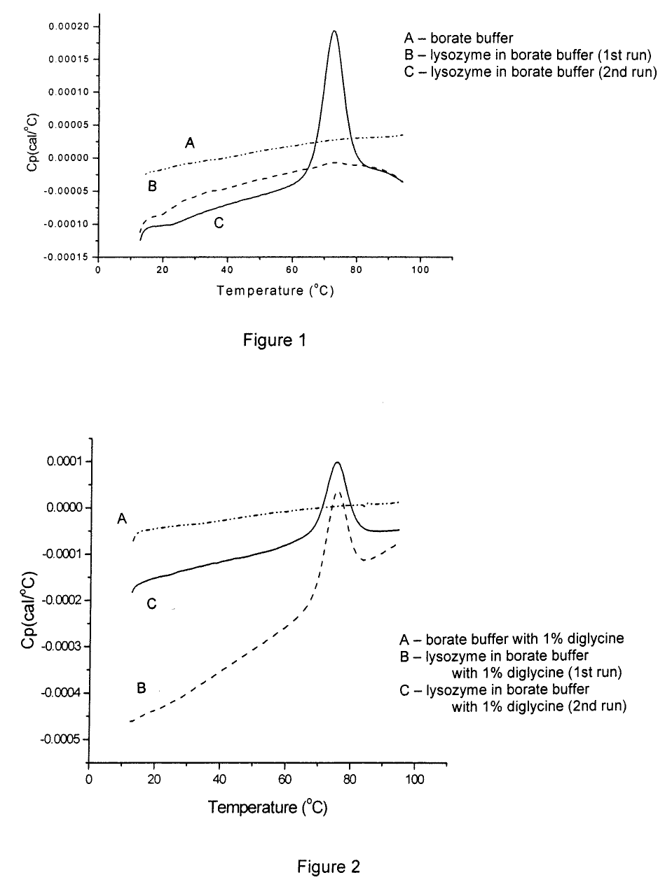

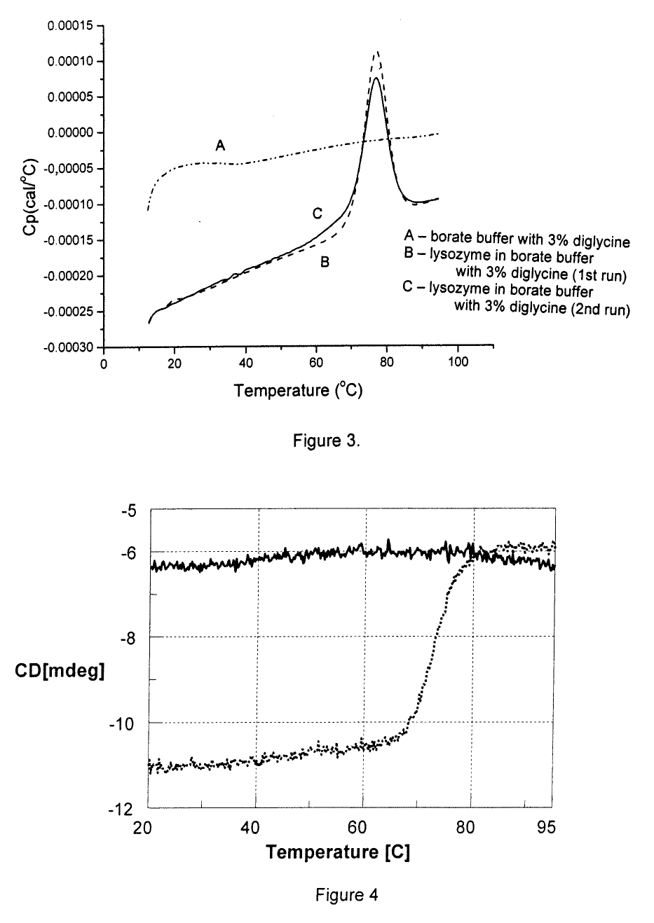

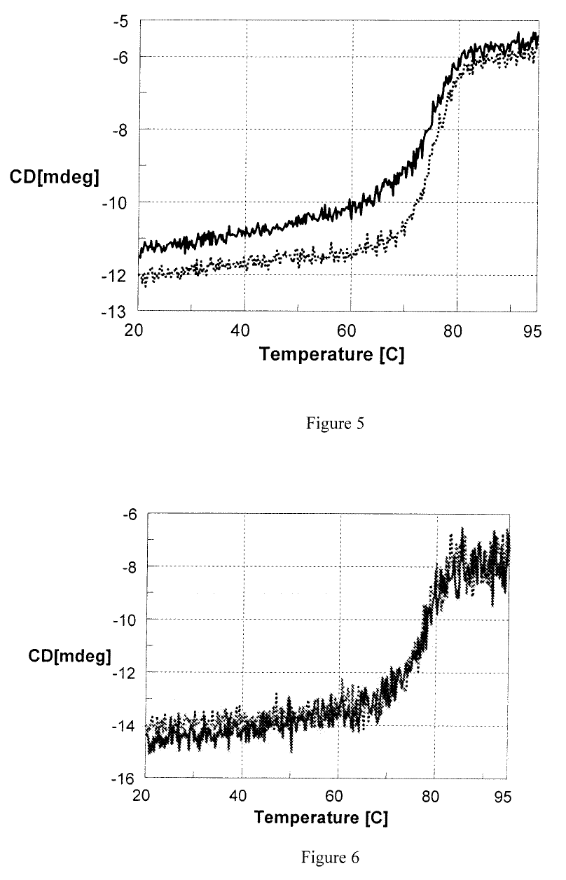

The graph of FIG. 1 shows that the unfolded lysozyme was not able to refold in the absence of diglycine, possibly due to an exothermic event or aggregation as indicated by the downward curved observed after the melting transition. In contrast, the temperature-induced denaturation was found to be at least partially reversible in the presence of diglycine although the values of ΔH were smaller in the second run than in the first run. The results illustrate the stabilizing effect of diglycine on lysozyme. Also, as shown in FIG. 4, only a sharp transition at Tm was obtained in the first run, implicating that the denaturation is not reversible in the absence of diglycine. On the other hand, the graphs of FIGS. 5 and 6 show that the denaturation is at least partially reversible for the lysozyme solutions formulated with diglycine as the first and second runs have similar denaturation curves.

In summary, the stabilizing effect of diglycine on the thermal unfolding of lysozyme as detected by DSC is consistent with the CD results. Both methods demonstrate that the diglycine not only improved the conformational stability of the lysozyme in the native form, but also increased the tendency of lysozyme to refold back to the native form upon unfolding.

### Experiment No. 2. Screen the Additive Glycerin, Trehalose, and Hyaluronic Acid (HA) in Combination with 1% Diglycine, for Their Ability to Stabilize the Conformation of Lysozyme

A second experiment screened the additives glycerin, trehalose, and hyaluronic acid (HA) in combination with 1% diglycine, for their ability to stabilize the conformation of lysozyme. VP-DSC and CD were again used to measure the melting temperatures (Tm), unfolding enthalpies (ΔH), and the ability to refold the lysozyme upon unfolding.

Before DSC and CD analysis, the lysozyme solution was prepared in a borate buffer, pH 7.3 (sodium borate, 0.09%; boric acid, 0.85%; sodium chloride, 0.22%) at a concentration of 5 mg/mL. It was then dialyzed against the formulated solutions containing either 1% glycerin, 1% trehalose, 5% trehalose, 1% diglycine and 3% glycerin, or 1% diglycine and 3% trehalose, at 2-8° C. for 16 hours. For the hyaluronic acid formulation, the lysozyme solution was dialyzed against the borate buffer, and subsequently diluted to the desired concentrations with 0.2% of hyaluronic acid for analyses. Thermal scans were performed twice to investigate the reversibility of thermal unfolding for both methods.

All DSC thermograms were obtained on a VP-DSC (Microcal, USA) at a scan rate of 90° C. per hour from 10° C. to 95° C. All samples were diluted with the formulated solutions to a concentration of 0.5 mg/mL, and degassed for 4 minutes using a vacuum. The thermograms were corrected for the baseline signal and normalized for protein concentration. Using a non-2-state unfolding model, the melting temperatures Tm and the unfolding enthalpies (ΔH) were determined using Origin software.

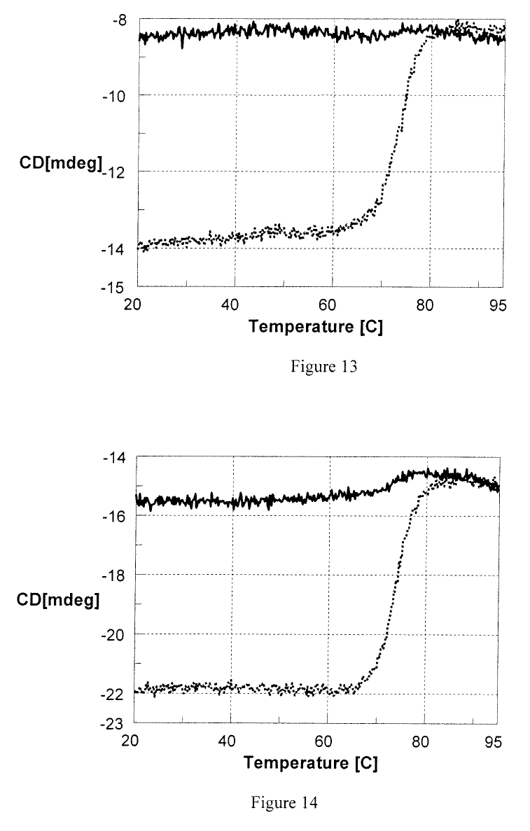

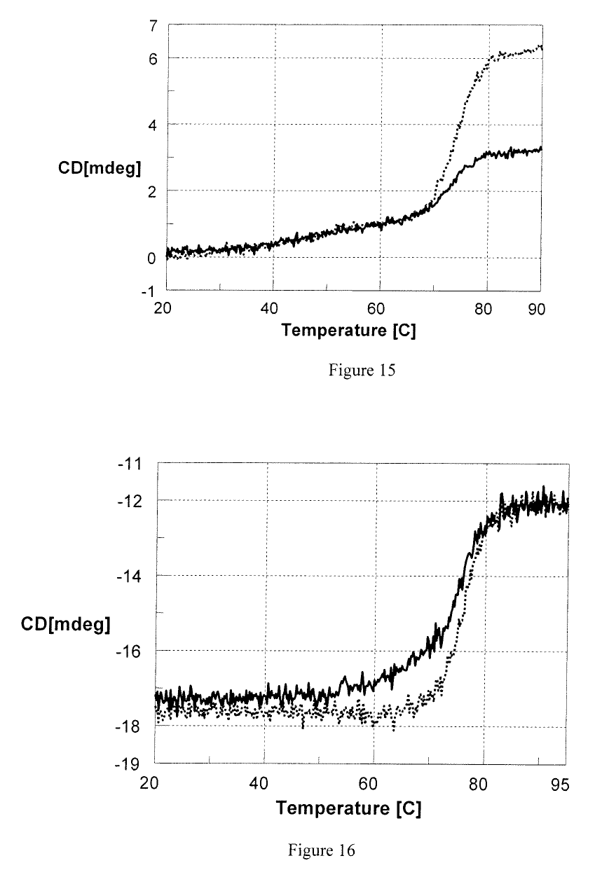

The VP-DSC results are supported by the circular dichroism (CD) spectral measurements which were conducted as a function of temperature on a Jasco-810 Spectropolarimeter. The far-UV CD studies were performed in a 1-mm pathlength cell at a protein concentration of 0.2 mg/mL, and a scan rate of 1° C./min form 20° C. to 95° C. The melting temperatures and the Van't Hoff enthalpies were determined from denaturation curves obtained from the mean residue ellipticity at 230 nm as a function of temperature. Tables 2 and 3 summarize the results of the CD measurements and FIGS. 13-18 show the CD-thermal scans.

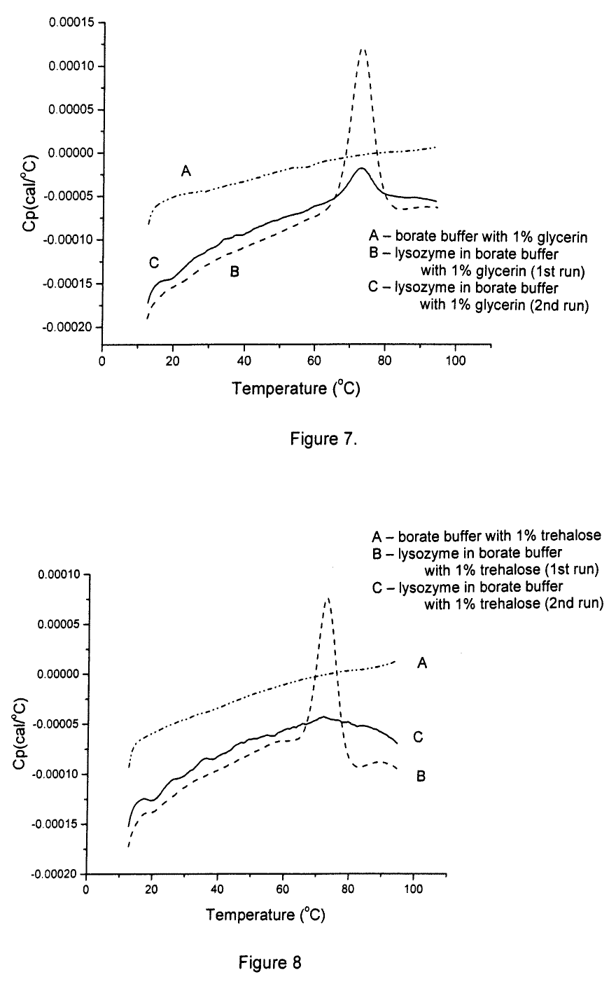

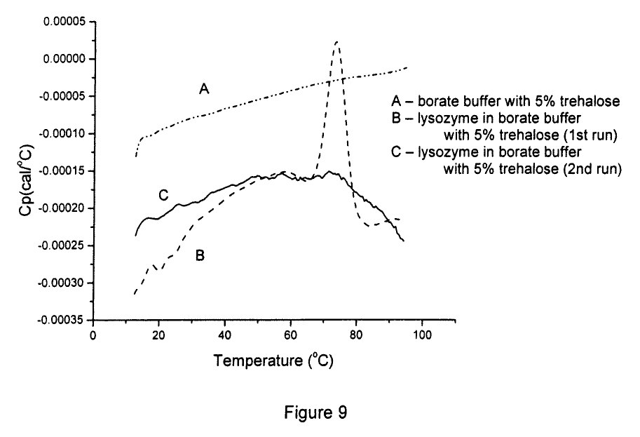

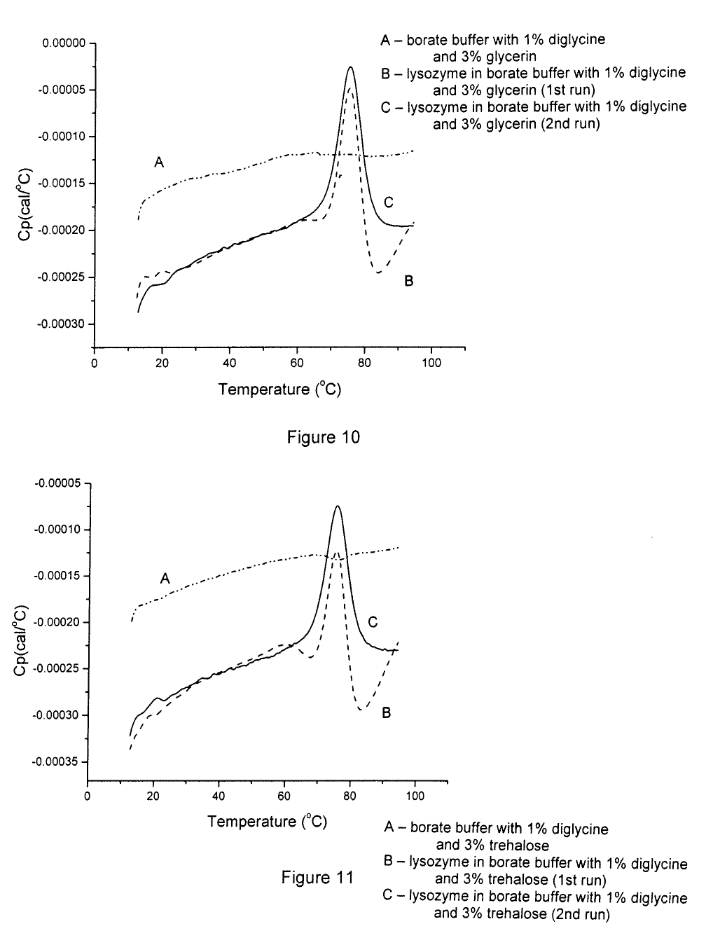

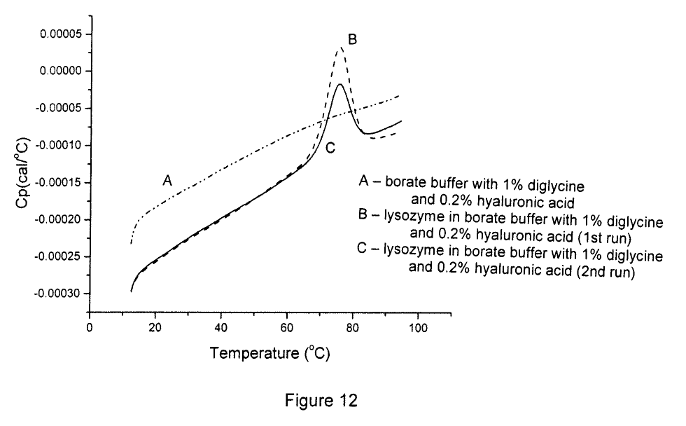

The results of the experiments are summarized in Tables 2 and 3, provided below and the DSC thermograms are provided in FIGS. 7-12.

TABLE 2
VP-DSC and CD Results of Lysozyme Formulated with One Additive

| | VP-DSC | | CD | | |
|---|---|---|---|---|---|
| | ΔH | Tm | ΔH | Tm | |
| Lysozyme Formulation Solutions | (1 × 10⁵ Cal/Mol) | (° C.) | (1 × 10⁵ Cal/Mol) | (° C.) | pH |
| Borate Buffer - 1^{st} run | 1.106 | 72.43 | 1.125 | 72.95 | 7.3 |
| Borate Buffer - 2^{nd} run | N/A | N/A | N/A | N/A | |
| Borate Buffer with 1% Diglycine - 1^{st} run | 1.112 | 74.93 | 1.057 | 75.15 | 6.8 |
| Borate Buffer with 1% Diglycine - 2^{nd} run | 0.08694 | 74.84 | 0.7601 | 75.13 | |
| Borate Buffer with 3% Diglycine - 1^{st} run | 1.091 | 76.67 | 1.164 | 77.05 | 6.4 |
| Borate Buffer with 3% Diglycine - 2^{nd} run | 0.8811 | 76.52 | 0.8608 | 77.63 | |
| Borate Buffer with 1% Trehalose - 1^{st} run | 0.6384 | 72.81 | 1.101 | 73.65 | 7.1 |
| Borate Buffer with 1% Trehalose - 2^{nd} run | N/A | N/A | N/A | N/A | |
| Borate Buffer with 5% Trehalose - 1^{st} run | 0.6618 | 73.64 | 1.222 | 73.8 | 7 |
| Borate Buffer with 5% Trehalose - 2^{nd} run | N/A | N/A | N/A | N/A | |
| Borate Buffer with 1% Glycerin - 1^{st} run | 0.9262 | 72.84 | 1.104 | 74 | 6.8 |
| Borate Buffer with 1% Glycerin - 2^{nd} run | 0.1592 | 72.49 | 0.8943 | 73.2 | |

pH = pH of the solution

TABLE 3
VP-DSC and CD Results of Lysozyme Formulated
with 1% Diglycine and an Additive

| | VP-DSC | | CD | | |
|---|---|---|---|---|---|
| | ΔH | Tm | ΔH | Tm | |
| Lysozyme Formulation Solutions | (1 × 10⁵ Cal/Mol) | (° C.) | (1 × 10⁵ Cal/Mol) | (° C.) | pH |
| Borate Buffer - 1^{st} run | 1.106 | 72.43 | 1.125 | 72.95 | 7.3 |
| Borate Buffer - 2^{nd} run | N/A | N/A | N/A | N/A | |
| Borate Buffer with 1% Diglycine - 1^{st} run | 1.112 | 74.93 | 1.057 | 75.15 | 6.8 |
| Borate Buffer with 1% Diglycine - 2^{nd} run | 0.08694 | 74.84 | 0.7601 | 75.13 | |
| Borate Buffer with 1% Diglycine + 3% | 0.9216 | 75.23 | 1.1101 | 77.95 | 6.1 |
| Glycerin - 1^{st} run | | | | | |
| Borate Buffer with 1% Diglycine + 3% | 0.7951 | 75.14 | 0.6820 | 75.6 | |
| Glycerin - 2^{nd} run | | | | | |
| Borate Buffer with 1% Diglycine + 3% | 0.9499 | 75.15 | 1.1264 | 77.15 | 6.6 |
| Trehalose - 1^{st} run | | | | | |
| Borate Buffer with 1% Diglycin + 3% | 0.7538 | 75.1 | 0.6828 | 76.99 | |
| Trehalose - 2^{nd} run | | | | | |
| Borate Buffer with 1% Diglycine + 0.2% | 0.8428 | 74.93 | 0.8953 | 75.69 | 7.1 |
| Hyaluronic Acid - 1^{st} run | | | | | |
| Borate Buffer with 1% Diglycine + 0.2% | 0.5188 | 74.94 | 0.7765 | 76.86 | |
| Hyaluronic Acid - 2^{nd} run | | | | | |

pH = pH of the solution

As shown in the VP-DSC results of Table 2, the Tm did not increase with increasing concentrations of glycerin and trehalose. Also, as shown in FIGS. 7-8, only very small peaks or no peaks were observed for the second scan of lysozyme when formulated with glycerin and trehalose, respectively. This indicates that the unfolded lysozyme was not able to refold when using glycerin and trehalose alone in the formulations. However, when the formulations included 1% diglycine, the temperature induced denaturation was found to be at least partially reversible. As shown in the VP-DSC results in Table 3, the ΔH values of the reheated samples were 86 and 79%, respectively, compared to the ΔH values of the first scans of these formulations.

As for the formulations with hyaluronic acid (HA), the addition of 0.2% hyaluronic acid and 1% diglycine to the lysozyme solution yielded haziness, which implies that hyaluronic acid is not fully compatible with lysozyme. Nevertheless, the Tm in the DSC analysis on lysozyme in 0.2% hyaluronic acid and 1% diglycine is the same as the Tm with diglycine alone, but with a smaller peak, as indicated by a slightly lower enthalpy. The re-heated formulation of hyaluronic acid and diglycine had a ΔH which was 62% the area of the first scan, thereby indicating a significant level of reversibility.

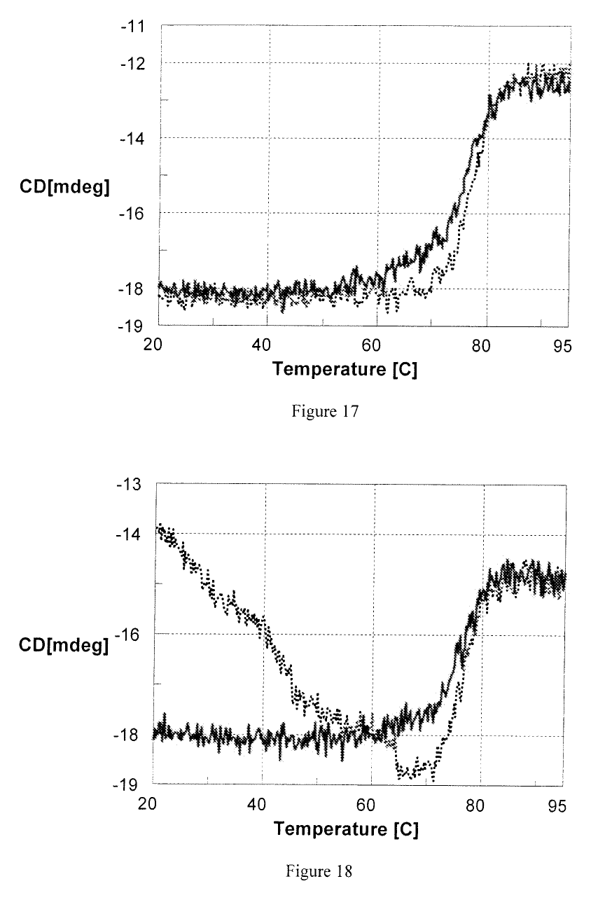

As can be seen in Table 2, when the CD spectra was measured as a function of temperature, glycerin (1%) and trehalose (1% and 5%) alone did not provide conformational stability to the lysozyme as the Tm values did not increase. Also, as can be seen in FIGS. 13-15, the CD data from the second scan showed that the lysozyme formulated with trehalose was not able to refold whereas when formulated with glycerin, a partial refolding phenomenon was observed. Table 3 and FIGS. 16 and 17 show that with the addition of 1% diglycine, the unfolded lysozyme was able to refold, revealing that the denaturation is at least partially reversible. This is consistent with the VP-DSC data.

The stabilizing effect of diglycine on the thermal unfolding of lysozyme as detected by DSC is comparable with the CD results. Both methods demonstrated that diglycine with the additives glycerin and trehalose improves the conformational stability of the lysozyme in the native form and increases the tendency of lysozyme to refold back to the native form after thermal unfolding.

Additional formulations of the inventive ophthalmic compositions include:

Formulation 3 - Ophthalmic Solution with Diglycine

| Ingredients | % w/w |
|---|---|
| Diglycine | 1.00 |
| Sodium Borate | 0.09 |
| Boric Acid | 0.85 |
| Sodium Chloride | 0.10 |
| Lysozyme | 0.50 |
| Potassium Chloride | 0.10 |
| Alexidine | 3 ppm |

Formulation 4 - Ophthalmic Solution with Diglycine

| Ingredients | % w/w |
|---|---|
| Diglycine | 1.00 |
| Sodium Borate | 0.09 |
| Boric Acid | 0.85 |
| Sodium Chloride | 0.10 |
| Lysozyme/Lactoferrin | 0.50 |
| Potassium Chloride | 0.10 |
| Alexidine | 3 ppm |

Formulation 5 - Ophthalmic Solution with Diglycine

| Ingredients | % w/w |
|---|---|
| Diglycine | 1.00 |
| Sodium Borate | 0.09 |
| Boric Acid | 0.85 |
| Sodium Chloride | 0.10 |
| Lactoferrin | 0.50 |
| Potassium Chloride | 0.10 |
| Alexidine | 3 ppm |

Formulation 6 - Ophthalmic Solution with Diglycine

| Ingredients | % w/w |
|---|---|
| Diglycine | 1.00 |
| Sodium Borate | 0.09 |
| Boric Acid | 0.85 |
| Sodium Chloride | 0.10 |
| Lysozyme | 0.50 |
| Potassium Chloride | 0.10 |
| Sorbic Acid/EDTA | 0.2/0.2 |

Formulation 7 - Ophthalmic Solution with Diglycine and Trehalose

| Ingredients | % w/w |
|---|---|
| Diglycine | 1.00 |
| Sodium Borate | 0.09 |
| Boric Acid | 0.85 |
| Sodium Chloride | 0.10 |
| Lysozyme | 0.50 |
| Trehalose | 0.20 |
| Potassium Chloride | 0.10 |
| Alexidine | 3 ppm |

Formulation 8 = Ophthalmic Solution with Diglycine and HA

| Ingredients | % w/w |
|---|---|
| Diglycine | 1.00 |
| Sodium Borate | 0.09 |
| Hyaluronic Acid | 0.10 |
| Boric Acid | 0.85 |
| Sodium Chloride | 0.10 |
| Lysozyme | 0.50 |
| Potassium Chloride | 0.10 |
| Benzalkonium Chloride (BAK) | 50-100 ppm |

Formulation 9 = Ophthalmic Solution with Diglycine

| Ingredients | % w/w |
|---|---|
| Diglycine | 1.00 |
| Sodium Borate | 0.20 |
| Boric Acid | 0.85 |
| Citric Acid | 0.05 |
| Sodium Chloride | 0.05 |
| Lysozyme | 0.50 |
| Potassium Chloride | 0.05 |
| Alexidine | 3 ppm |

Formulation 10 = Ophthalmic Solution with Diglycine, HA and Trehalose

| Ingredients | % w/w |
|---|---|
| Diglycine | 1.00 |
| Hyaluronic Acid | 0.05 |
| Trehalose | 3.00 |
| Sodium Borate | 0.09 |
| Boric Acid | 0.85 |
| Sodium Chloride | 0.02 |
| Lysozyme | 0.50 |
| Potassium Chloride | 0.02 |
| Sodium Perborate | 0.20 |

Formulation 11 = Ophthalmic Solution with Diglycine

| Ingredients | % w/w |
|---|---|
| Diglycine | 1.00 |
| Sodium Borate | 0.09 |
| Boric Acid | 0.85 |
| Sodium Chloride | 0.05 |
| Lysozyme | 0.50 |
| Potassium Chloride | 0.10 |
| Loteprednol Etabonate | 0.50 |
| Alexidine | 3 ppm |

In the foregoing formulations lysozyme and/or lactoferrin may be considered an optional component that supplements natural tear protein. Also the alexidine and/or loteprednol etabonate should be taken as illustrative of the inclusion of therapeutic agents generally.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

Lastly, those skilled in the art may recognize other equivalents to the specific embodiment described herein which equivalents are intended to be encompassed by the claims attached hereto.

1. An ophthalmic composition comprising an epithelium cell stabilizer component including at least one member of the group consisting of diglycine, triglycine, tetraglycine and pentaglycine.

2. A composition as in claim 1 wherein the epithelium cell stabilizer component includes diglycine.

3. A composition as in claim 1 wherein the epithelium cell stabilizer component is present in the composition in an amount of from about 0.01% to about 10% w/w.

4. A composition as in claim 1, further comprising a polyol component.

5. A composition as in claim 4 wherein said polyol component includes at least one member of the group consisting of glycerin, trehalose, arabitol, erythirtol, glycerol, lactitol, maltitol, mannitol, sorbitol, and xylitol.

6. A composition as in claim 4 wherein said polyol component is present in the composition in an amount of from 0.2% w/w to 3% w/w.

7. A composition as in claim 1, further comprising a buffering system selected from the group consisting of a borate buffer, a phosphate buffer, a carbonate buffer, a citrate buffer or a mixture two or more thereof.

8. A composition as in claim 1, further comprising at least one tear protein that is present in the composition in an amount of from 0.01% w/w to 10% w/w.

9. A composition as in claim 8 wherein the tear protein is selected from the group consisting of lysozyme, lactoferrin and mixtures thereof.

10. A composition as in claim 11 further comprising at least one polymer selected from the group consisting of carboxymethylcellulose, alginate, carbomer, hydroxypropyl methylcellulose, polyvinyl alcohol, polypropylene, ethylcellulose and povidone.

11. A composition as in claim 11 further comprising at least one preservative selected from the group consisting of benzalkonium chloride, benzalkonium chloride/ethylenediaminetetraacetic acid, sorbic acid/ethylenediaminetetraacetic acid, biguanides, sodium perborate and hydrogen peroxide.

12. A composition as in claim 11 further comprising a surfactant selected from the group consisting of polysorbate, cremophor, triton, poloxamer, poloxamine and tyloxapol.

13. A composition as in claim 11 further comprising at least one therapeutic agent selected from the group consisting of anti-inflammatory agents, antibiotics, immunosuppressive agent, antifungal agents and antiprotozoal agents.

14. A composition as in claim 1 having an osmolality from 200 mOsmol/kg to 400 mOsmol/kg.

15. A composition as in claim 1, further comprising hyaluronic acid or a physiologically compatible salt thereof.

16. A composition as in claim 1 formulated as an eye drop or eye wash solution.

17. A composition as in claim 1 formulated for cleaning, disinfecting and/or packaging contact lenses.

18. A method of treating or preventing dry eye comprising administering an effective amount of an ophthalmic composition to the eye, said composition comprising an epithelium cell stabilizer component including at least one member of the group consisting of diglycine, triglycine, tetraglycine and pentaglycine.

19. A method as in claim 18 wherein said composition further comprises at least one tear protein selected from the group consisting of lysozyme, lactoferrin and mixtures thereof, the total amount of the tear protein present in the composition in an amount of from 0.01% w/w to 10% w/w.

20. A method as in claim 18 wherein said composition further comprises a polyol selected from the group consisting of glycerin, trehalose, arabitol, erythirtol, glycerol, lactitol, maltitol, mannitol, sorbitol and xylitol, the total amount of the polyol present in the composition in an amount of from 0.2% w/w to 3% w/w.

21. A method as in claim 18 wherein the epithelium cell stabilizer component includes diglycine.
